(19) United States
(12) Patent Application Publication
(10) Pub. No.: US 2008/0110452 A1
(43) Pub. Date: May 15, 2008

(54) NEBULIZER AND METHOD FOR CONTROLLING AN AMOUNT OF LIQUID THAT IS ATOMIZED BY THE NEBULIZER

(75) Inventors: Paul T. Kotnik, Commerce Township, MI (US); David A. Ross, Columbiaville, MI (US)

Correspondence Address: CANTOR COLBURN, LLP, 20 Church Street, 22nd Floor, Hartford, CT, 06103, US

(73) Assignee: DELPHI TECHNOLOGIES INC., Troy, MI (US)

(21) Appl. No.: 11/560,130

(22) Filed: Nov. 15, 2006

(51) Int. Cl. A61M 11/00; G08B 13/14

(52) U.S. Cl. 128/200.14; 340/572.1

(57) ABSTRACT

A nebulizer and a method for controlling an amount of liquid that is atomized by the nebulizer is provided. The nebulizer determines an identity of an individual and accesses a remote computer to determine a desired amount of liquid to atomize for the individual.

### TECHNICAL FIELD

This application relates to a nebulizer and a method for controlling an amount of liquid that is atomized by the nebulizer.

### BACKGROUND

Nebulizers have been utilized to atomize a medicinal liquid that is received by an individual. However, the nebulizer does not have internal logic to determine how much of the medicinal liquid to atomize. As a result, the nebulizer may atomize an amount of liquid that is less than an amount prescribed by a physician. Alternately, the nebulizer may atomize an amount of liquid that is more than the amount prescribed by the physician.

Accordingly, the inventors herein have recognized a need for an improved nebulizer that minimizes and/or eliminates the foregoing deficiencies.

### SUMMARY OF THE INVENTION

A method for controlling an amount of liquid that is atomized by a nebulizer in accordance with an exemplary embodiment. The method includes determining an identity of an individual and a type of liquid contained in a container from either a radio frequency identifier (RFID) tag or a bar code associated with the container. The container is configured to be disposed on the nebulizer. The method further includes accessing a database on a remote computer utilizing a first communication interface device associated with the nebulizer to determine a desired amount of the liquid to atomize for the individual, based on the identity of the individual and the type of liquid in the container. The method further includes activating an atomizing member of the nebulizer that receives liquid from the container to atomize the desired amount of the liquid.

A nebulizer in accordance with another exemplary embodiment is provided. The nebulizer includes a container having an RFID tag or a bar code identifying an individual and a type of liquid contained in the container. The nebulizer further includes a sensor configured to read either the RFID tag or the bar code. The nebulizer further includes a communication interface device. The nebulizer further includes a controller operably communicating with the sensor and the communication interface device. The controller is configured to induce the communication interface device to communicate with a database on a remote computer to determine a desired amount of the liquid to atomize for the individual, based on an identity of the individual and the type of liquid in the container. The controller is further configured to activate an atomizing member receiving liquid from the container to atomize the desired amount of the liquid.

A method for monitoring an amount of liquid that is atomized by a nebulizer in accordance with another exemplary embodiment. The method includes determining an amount of a liquid atomized by an atomizing member of the nebulizer. The method further includes sending a message from the nebulizer to a remote computer utilizing a communication interface device. The message indicates the amount of liquid atomized by the atomizing member. The method further includes updating a record in a database to indicate the amount of liquid atomized by the atomizing member, utilizing the remote computer.

A system for monitoring an amount of liquid that is atomized in accordance with another exemplary embodiment. The system includes a nebulizer having a controller and a communication interface device operably communicating with the controller. The controller is configured to determine an amount of the liquid atomized by an atomizing member of the nebulizer. The controller is further configured to induce the communication interface device to send a message indicating the amount of liquid atomized by the atomizing member. The system further includes a remote computer configured to receive the message and to update a record in a database to indicate the amount of liquid atomized by the atomizing member.

### BRIEF DESCRIPTION OF THE DRAWINGS

FIG. 1 is a block diagram of a system for controlling an amount of liquid that is atomized and for monitoring an amount of liquid that is atomized in accordance with an exemplary embodiment; and

FIGS. 2-4 are flowcharts of a method for controlling an amount of liquid that is atomized and for monitoring an amount of liquid that is atomized in accordance with another exemplary embodiment.

### DESCRIPTION OF EXEMPLARY EMBODIMENTS

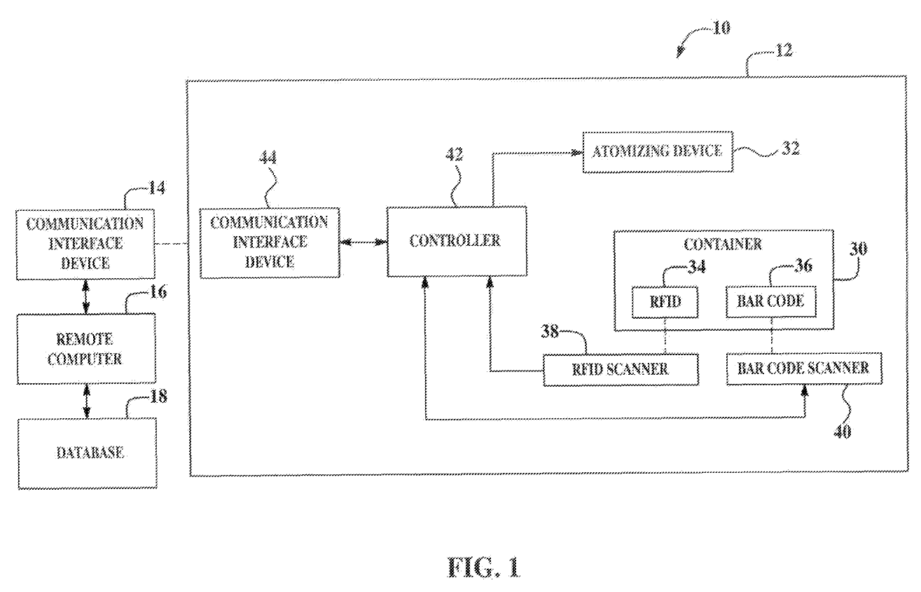

Referring to FIG. 1, a system 10 for controlling an amount of liquid that is atomized and for monitoring an amount of liquid that is atomized is provided. The system 10 includes a nebulizer 12, a communication interface device 14, a remote computer 16, and a database 18.

The nebulizer 12 is provided to atomize a liquid stored therein. The nebulizer 12 includes a container 30, an atomizing device 32, an RFID 34, a bar code 36, an RFID scanner 38, a bar code scanner 40, a controller 42, and a communication interface device 44.

The container 30 is provided to hold a liquid therein and is in fluid communication with the atomizing device 32. The atomizing device 32 atomizes at least a portion of the liquid in response to receiving a control signal from the controller 42.

The RFID 34 is provided to transmit an RF signal that indicates (i) the identity of the individual associated with the nebulizer 12, (ii) a nebulizer identifier, and (iii) the type of liquid contained in the container 30. In one exemplary embodiment, the RFID 34 is disposed on the container 30. Of course, in other exemplary embodiments, the RFID 34 can be disposed on other locations on the nebulizer 12.

The RFID scanner 38 is provided to receive the RF signals transmitted by the RFID 34 and to send a message having data contained in the RF signals to the controller 42. The RFID scanner 38 is disposed in a relatively close proximity to the RFID 34.

The bar code 36 contains data that indicates (i) the identity of the individual associated with the nebulizer 12, (ii) a nebulizer identifier, and (iii) the type of liquid contained in the container 30. In one exemplary embodiment, the bar code 36 is disposed on the container 30. Of course, in other exemplary embodiments, the bar code 36 can be disposed on other locations on the nebulizer 12.

The bar code scanner 40 is provided to read data contained on the bar code 36 and to send a message having the data to the controller 42. The bar code scanner 40 is disposed in a relatively close proximity to the bar code 36.

The controller 42 is provided to control the operation of the nebulizer 12. The controller 42 is operably coupled to the atomizing device 32, the RFID scanner 38, the bar code scanner 40, and the communication interface device 44. The controller 42 is configured to generate a signal that induces the atomizing device 32 to atomize liquid. The controller 42 is further configured to receive messages from the RFID scanner 38 and the bar code scanner 40 having data indicating the identity of an individual, the nebulizer identifier or ID, and the type of liquid in the container 30. The controller 42 is further configured to receive messages from the communication interface device 44 and to send messages to the communication interface device 44.

The communication interface device 44 is provided to transmit messages to the remote communication interface device 14 and to receive messages from the device 14. In one exemplary embodiment, the device 44 is an RF transceiver that transmits RF signals having messages contained therein to the device 14 and receives RF signals having messages contained therein from the device 14. In another exemplary embodiment, the device 44 is a signal repeater that is hardwired to the device 14 and sends the messages to the device 14 and receives messages from the device 14. The device 44 is electrically coupled to the controller 42.

The communication interface device 14 is provided to transmit messages to the remote communication interface device 44 and to receive messages from the device 44. In one exemplary embodiment, the device 14 is an RF transceiver that transmits RF signals having messages contained therein to the device 44 and receives RF signals having messages contained therein from the device 14. In another exemplary embodiment, the device 14 is a signal repeater that is hardwired to the remote device 44 and sends the messages to the device 44 and receives message from the device 44. The communication interface device 14 is electrically coupled to the remote computer 16.

The remote computer 16 is provided to access records in the database 18 to determine a record associated with an individual. The record has at least the following data: a name of the individual, a nebulizer identifier associated with a nebulizer assigned to the individual, a type of liquid identifier, and a value indicating a desired amount of liquid to atomize. The record can also have time and date information. The remote computer 16 is further provided to update a record associated with the individual to indicate the amount of liquid atomized by the nebulizer. The remote computer 16 is operably coupled between the communication interface device 14 and the database 18.

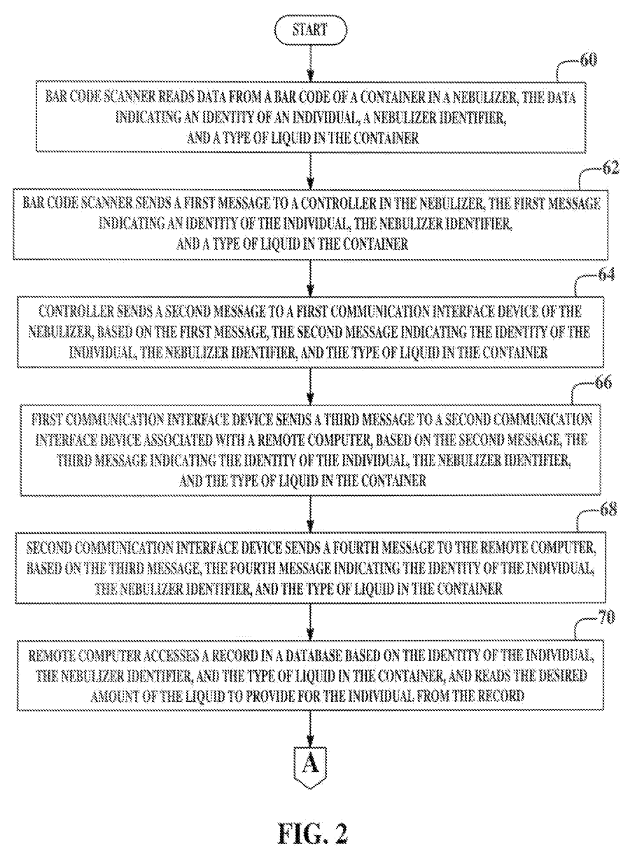

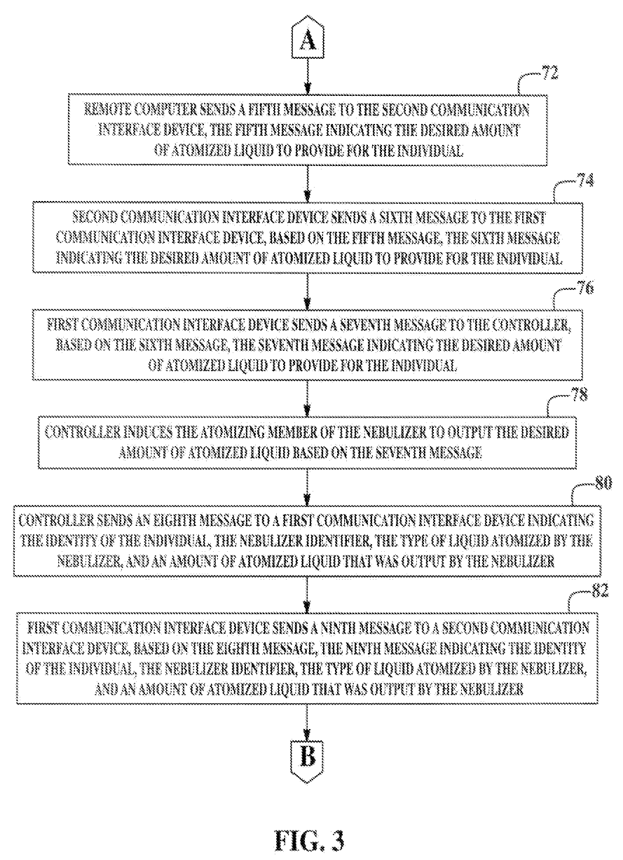

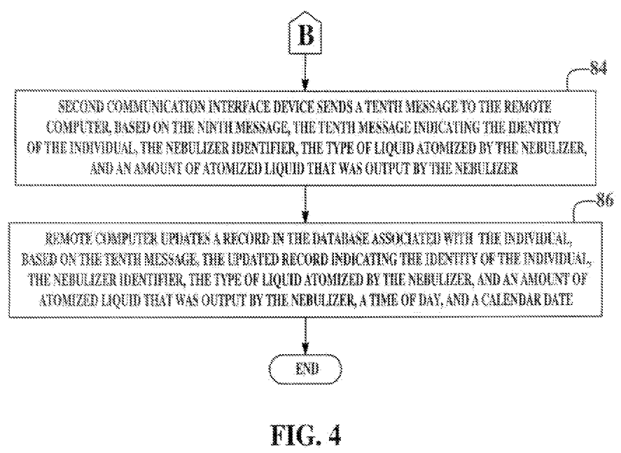

Referring to FIGS. 2-4, a flowchart of a method for controlling an amount of liquid that is atomized and for monitoring an amount of liquid that is atomized will now be explained. The method can be implemented utilizing the system 10. It should be noted that in the exemplary method, the bar code scanner 40 and the bar code 36 is utilized to determine an identity of an individual, the nebulizer identifier, and a type of liquid in the container 30. In an alternative embodiment, the RFID scanner 38 and the RFID could be used to determine the identity of the individual and the type of liquid in the container 30.

At step 60, the bar code scanner 40 reads data from the bar code 36 of the container 30 in the nebulizer 12. The data indicates an identity of an individual, a nebulizer identifier, and a type of liquid in the container 30.

At step 62, the bar code scanner 40 sends a first message to the controller 42 in the nebulizer 12. The first message indicates the identity of the individual, the nebulizer identifier, and the type of liquid in the container 30.

At step 64, the controller 42 sends a second message to the communication interface device 44 of the nebulizer 12, based on the first message. The second message indicates the identity of the individual, the nebulizer identifier, and the type of liquid in the container 30.

At step 66, the communication interface device 44 sends a third message to the communication interface device 14 associated with the remote computer 16, based on the second message. The third message indicates the identity of the individual, the nebulizer identifier, and the type of liquid in the container 30.

At step 68, the communication interface device 14 sends a fourth message to the remote computer 16, based on the third message. The fourth message indicates the identity of the individual, the nebulizer identifier, and the type of liquid in the container 30.

At step 70, the remote computer 16 accesses a record in the database 18 based on the identity of the individual, the nebulizer identifier, and the type of liquid in the container 30, and reads the desired amount of the liquid to provide for the individual from the record.

At step 72, the remote computer 16 sends a fifth message to the second communication interface device 14. The fifth message indicates the desired amount of atomized liquid to provide for the individual.

At step 74, the communication interface device 14 sends a sixth message to the communication interface device 44, based on the fifth message. The sixth message indicates the desired amount of atomized liquid to provide for the individual.

At step 76, the communication interface device 44 sends a seventh message to the controller 42, based on the sixth message. The seventh message indicates the desired amount of atomized liquid to provide for the individual.

At step 78, the controller 42 induces the atomizing member 32 of the nebulizer 12 to output the desired amount of atomized liquid, based on the seventh message. It should be noted that the controller 42 can calculate the amount of atomized liquid based on a stored value indicative of an amount of liquid that can be atomized per unit time by the atomizing member 32 and a measured time that the atomizing member 32 is atomizing the liquid. In particular, the amount of atomized liquid is calculated by multiplying the stored value indicative of the amount of liquid that can be atomized per unit time by the atomizing member 32, with the measured time that the atomizing member 32 is atomizing the liquid.

At step 80, the controller 42 sends an eighth message to the communication interface device 44 indicating the identity of the individual, the nebulizer identifier, the type of liquid atomized by the nebulizer, and an amount of atomized liquid that was output by the nebulizer 12.

At step 82, the communication interface device 44 sends a ninth message to the communication interface device 14, based on the eighth message. The ninth message indicating the identity of the individual, the nebulizer identifier, the type of liquid atomized by the nebulizer 12, and an amount of atomized liquid that was output by the nebulizer 12.

At step 84, the communication interface device 14 sends a tenth message to the remote computer 16, based on the ninth message. The tenth message indicates the identity of the individual, the nebulizer identifier, the type of liquid atomized by the nebulizer 12, and an amount of atomized liquid that was output by the nebulizer 12.

At step 86, the remote computer 16 updates a record in the database 18 associated with the individual, based on the tenth message. The updated record indicates the identity of the individual, the nebulizer identifier, the type of liquid atomized by the nebulizer 12, and an amount of atomized liquid that was output by the nebulizer, a time of day, and a calendar date. After step 86, the method is exited.

The above-described method can be embodied in the form of computer-implemented software algorithms and apparatuses for practicing those processes. In an exemplary embodiment, the method is embodied in computer program code executed by one or more elements of the system 10. The present method may be embodied in the form of computer program code containing instructions stored in tangible media, such as floppy diskettes, CD-ROMs, hard drives, flash memory, or any other computer-readable storage medium, wherein, when the computer program code is loaded into and executed by a computer or controller, the computer or controller becomes an apparatus for practicing the invention.

The nebulizer and the method for controlling an amount of liquid that is atomized represents a substantial improvement over other nebulizers and methods. In particular, the nebulizer and the method provide a technical effect of determining an identity of an individual and accessing a remote computer to determine a desired amount of liquid to atomize for the individual.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed for carrying out this invention, but that the invention will include all embodiments falling within the scope of the appended claims. Moreover, the use of the terms first, second, etc. do not denote any order or importance, but rather the terms first, second, etc. are used to distinguish one element from another. Further, the use of the terms a, an, etc. do not denote a limitation of quantity, but rather denote the presence of at least one of the referenced item.

What is claimed is:

1. A method for controlling an amount of liquid that is atomized by a nebulizer, comprising:
determining an identity of an individual and a type of liquid contained in a container from either an RFID tag or a bar code associated with the container, the container configured to be disposed on the nebulizer;
accessing a database on a remote computer utilizing a first communication interface device associated with the nebulizer to determine a desired amount of the liquid to atomize for the individual, based on the identity of the individual and the type of liquid in the container; and
activating an atomizing member of the nebulizer that receives liquid from the container to atomize the desired amount of the liquid.

2. The method as in claim 1, wherein determining the identity of the individual, an identity of the nebulizer, and the type of the liquid contained in the container comprises reading data on the bar code utilizing a bar code scanner, the data indicating the identity of the individual, a nebulizer identifier, and the type of the liquid contained in the container.

3. The method as in claim 1, wherein determining the identity of the individual and the type of the liquid contained in the container comprises reading data on the RFID tag utilizing a RFID scanner, the data indicating the identity of the individual and the type of the liquid contained in the container.

4. The method as in claim 1, wherein accessing the database on a remote computer comprises:
sending a first message from a controller associated with the nebulizer to the first communication interface device, the first message indicating the identity of the individual, a nebulizer identifier, and the type of the liquid contained in the container;
sending a second message from the first communication device to a second communication device associated with the remote computer, the second message indicating the identity of the individual and the type of the liquid contained in the container based on the first message;
accessing a record in the database utilizing the remote computer based on the identity of the individual, the nebulizer identifier, and the type of the liquid contained in the container;
reading the desired amount of the liquid to provide for the individual, utilizing the remote computer;
sending a third message from the remote computer to the second communication interface device, the third message indicating the desired amount of the liquid to provide for the individual;
sending a fourth message from the second communication interface device to the first communication interface device, the fourth message indicating the desired amount of the liquid to provide for the individual based on the third message; and
sending a fifth message from the first communication interface device to the controller, the fifth message indicating the desired amount of the liquid to provide for the individual based on the fourth message.

5. The method as in claim 1, wherein the liquid is a medicinal liquid.

6. A nebulizer, comprising:
a container having an RFID tag or a bar code identifying an individual and a type of liquid contained in the container;
a sensor configured to read either the RFID tag or the bar code;
a communication interface device; and
a controller operably communicating with the sensor and the communication interface device, the controller configured to induce the communication interface device to communicate with a database on a remote computer to determine a desired amount of the liquid to atomize for the individual, based on an identity of the individual and the type of liquid in the container, the controller further configured to activate an atomizing member receiving liquid from the container to atomize the desired amount of the liquid.

7. The nebulizer of claim 6, wherein the sensor is a bar code scanner configured to read data from the bar code that identifies the individual and the type of liquid contained in the container.

8. The nebulizer of claim 6, wherein the sensor is an RFID scanner configured to receive signals from the RFID having data identifying the individual and the type of liquid contained in the container.

9. The nebulizer of claim 6, wherein the communication interface device comprises an RF transceiver configured to transmit RF signals.

10. The nebulizer of claim 6, wherein the liquid is a medicinal liquid.

11. A method for monitoring an amount of liquid that is atomized by a nebulizer, comprising:
determining an amount of a liquid atomized by an atomizing member of the nebulizer;
sending a message from the nebulizer to a remote computer utilizing a communication interface device, the message indicating the amount of liquid atomized by the atomizing member; and
updating a record in a database to indicate the amount of liquid atomized by the atomizing member, utilizing the remote computer.

12. The method as in claim 11, wherein the step of updating a record in the database further comprises updating the record to indicate a time of day and a calendar date that the atomizing member atomizes the liquid.

13. A system for monitoring an amount of liquid that is atomized, comprising:
a nebulizer having a controller and a communication interface device operably communicating with the controller, the controller configured to determine an amount of the liquid atomized by an atomizing member of the nebulizer, the controller further configured to induce the communication interface device to send a message indicating the amount of liquid atomized by the atomizing member; and
a remote computer configured to receive the message and to update a record in a database to indicate the amount of liquid atomized by the atomizing member.

14. The system of claim 13, wherein the remote computer is further configured to update the record in the database to indicate a time of day and a calendar date that the atomizing member atomizes the liquid.

15. The system of claim 13, wherein the liquid is a medicinal liquid.
